(19) United States
(12) Patent Application Publication
(10) Pub. No.: US 2022/0092764 A1
(43) Pub. Date: Mar. 24, 2022

(54) Low-Cost System for Inspecting the Integrity of a Wheel Rim

(71) Applicant: Eclectic Services Company (Pty) Ltd, Cape Town (ZA)

(72) Inventor: Alex Johan Botha, Cape Town (ZA)

(21) Appl. No.: 17/222,964

(22) Filed: Apr. 5, 2021

(30) Foreign Application Priority Data
Sep. 18, 2020 (ZA) 2020/05791

(51) Int. Cl. G06T 7/00; H04N 5/247; H04N 7/18; G06K 9/22; G01N 23/046; G01N 23/083; G01N 23/18

(52) U.S. Cl. CPC G06T 7/0004; H04N 5/247; H04N 7/181; G06K 9/228; G06T 2207/30136; G01N 23/083; G01N 23/18; G06T 2207/10116; G06T 2207/30152; G01N 23/046

(57) ABSTRACT

According to an aspect of the invention, there is provided a low-cost system for inspecting the integrity of a wheel rim, said system including one or more of the following: a portable compact x-ray device; a wheel gripping means operable to rotate and hold the rim in position; and an x-ray detector panel; wherein said x-ray detector panel is operable to produce an image of the wheel rim on a computer, to thereby manipulate and store images of said wheel rim.

### FIELD OF THE INVENTION

The present invention relates to a low-cost system for inspecting the integrity of a wheel rim.

### BACKGROUND TO THE INVENTION

Generally, a wheel rim is known as the outer edge of a wheel which holds the wheel or “tire” in place. More specifically, it makes up the outer circular design of the wheel on which the inside edge of the tire is mounted on vehicles.

For example, on a bicycle wheel the rim is a large hoop attached to the outer ends of the spokes of the wheel that holds the tire and tube. In cross-section, the rim is deep in the center and shallow at the outer edges, thus forming a “U” shape that supports for the bead of the tire casing.

In the automotive industry, alloy wheels are wheels that are made from an alloy of aluminium or magnesium. Alloys are mixtures of a metal and other elements. They generally provide greater strength over pure metals, which are usually much softer and more ductile.

It will be appreciated that in accordance with the conventional operation of a motor vehicle, a wheel rim of the vehicle may be damaged. This generally takes place when the vehicle is manoeuvred through treacherous terrain such as an unequal road surface including a ditch and/or vertical deflection mechanisms such as a speed hump or rocks, etc. Also, when the vehicle mounts a curb or the like, damage to the wheel rim may result.

In terms of the detection of damage to a rim wheel, the damage may not be readily apparent to the naked eye. In such instances, X-ray (DR) and computed tomography (CT) inspection systems are often used to provide damage detection and quality assurance processes in the automotive industry. In terms of this existing technology, the potentially damaged wheel rims are checked against conventional structural requirements and safety regulations to provide assurance regarding the structural integrity of the wheel rim.

In terms of existing technology in this industry, an X-ray inspection system is available which includes a manoeuvrable arm, with a portable compact X-ray machine that is mounted on the one side of the device and a detector on the other. In respect of this particular system, the swing arm is provided in the form of an image intensifier and a portable compact x-ray machine is added.

It will be appreciated by those in the industry, that the use of an image intensifier is out-dated and newer technologies have been developed which execute the same function with higher degrees of accuracy and precision.

### OBJECT OF THE INVENTION

It is an object of the invention to provide a low-cost system for inspecting the integrity of a wheel rim which provides for the advantages and addresses at least some of the issues and/or deficiencies described above.

### SUMMARY OF THE INVENTION

According to an aspect of the invention, there is provided a low-cost system for inspecting the integrity of a wheel rim, said system including one or more of the following:

a portable compact x-ray device;

a wheel gripping means operable to rotate and hold the rim in position; and an x-ray detector panel;

wherein said x-ray detector panel is operable to produce an image of the wheel rim on a computer, to thereby manipulate and store images of said wheel rim.

In an embodiment of the invention, said x-ray detector panel is operable to enable a user to check the integrity of one or more welding joints on the wheel rim made during repair of the wheel rim and/or checking for cracks in the wheel rim not visible the naked eye.

In an embodiment of the invention, said system further includes box operable to protect a user from radiation. In an example embodiment, said box is provided in the example form of a lead lined box.

In another embodiment of the invention, said system further includes one or more Closed Circuit Television (CCTV) cameras positioned in said box, wherein said cameras are operable to view the wheel rim turning around in the wheel gripping means.

In an embodiment of the invention, said x-ray scanning device is provided in the example form of a small portable device.

### BRIEF DESCRIPTION OF THE DRAWINGS

The present invention will now be described, by way of example only, with reference to the accompanying drawings, in which:

FIG. 1 shows a schematic diagram of a protective box of a system for inspecting the integrity of a wheel rim, in accordance with one or more embodiments of the invention;

FIG. 2 shows a schematic diagram of a plan view of the wheel gripping device of a system for inspecting the integrity of a wheel rim system, in accordance with one or more embodiments of the invention;

FIG. 3 shows a schematic diagram of a 3-dimensional view of the wheel gripping device of a system for inspecting the integrity of a wheel rim system, in accordance with one or more embodiments of the invention; and

FIG. 4 shows a schematic diagram of a plan view of an x-ray detector panel, in accordance with one or more embodiments of the invention.

### DESCRIPTION OF PREFERRED EMBODIMENT

The following description of the invention is provided as an enabling teaching of the invention. Those skilled in the relevant art will recognize that many changes can be made to the embodiment described, while still attaining the beneficial results of the present invention. It will also be apparent that some of the desired benefits of the present invention can be attained by selecting some of the features of the present invention without utilizing other features. Accordingly, those skilled in the art will recognize that modifications and adaptations to the present invention are possible and can even be desirable in certain circumstances and are a part of the present invention. Thus, the following description is provided as illustrative of the principles of the present invention and not a limitation thereof.

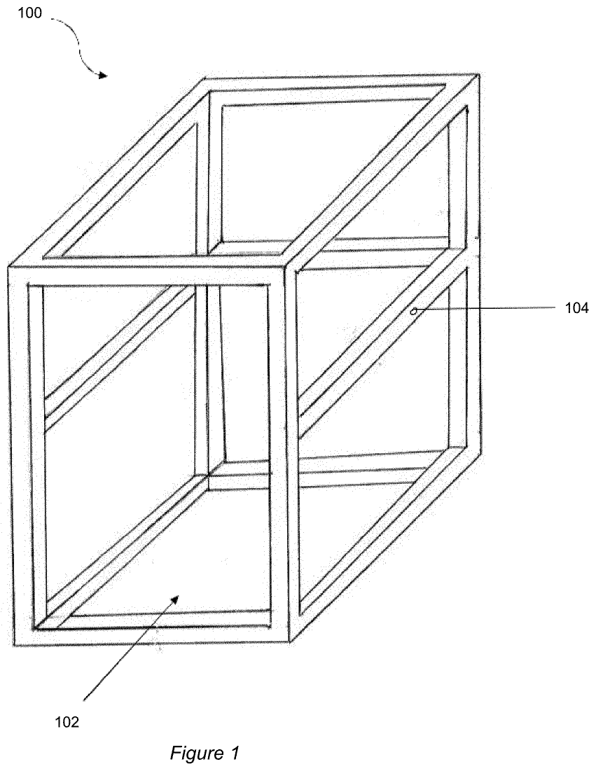

In FIG. 1, a protective box of a system for inspecting the integrity of a wheel rim a system for installing electrical wiring is generally described with reference to numeral 100.

The system 100 includes a doorway with a door 102 operable to open and close, in use.

The system 100 defines a hole 104 which can receive the shaft for the x-ray device of the system.

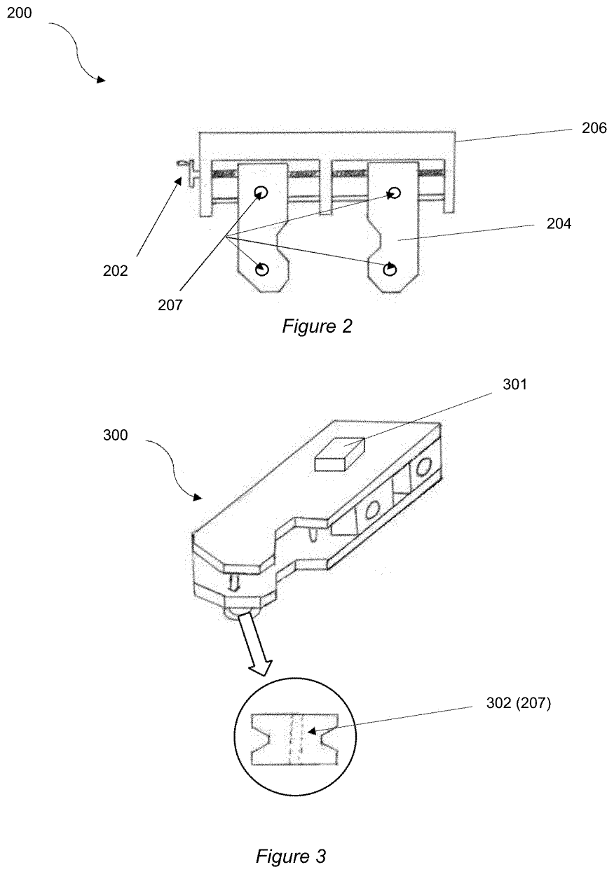

In FIG. 2, a schematic diagram of a plan view of the wheel gripping device of a system for inspecting the integrity of a wheel rim system is generally described with reference to numeral 200.

The device 200 includes a handle 202 for open and closing the wheel gripping device. The device 200 includes a set of pinchers 204 to hold the wheel rim in place, in use. The device 200 includes a back-end panel 206 which holds the pinchers in place.

The device 200 defines several holes in which a roller 207 can be received.

In FIG. 3, a schematic diagram of a 3-dimensional view of the wheel gripping device of a system for inspecting the integrity of a wheel rim system, is generally described with reference to numeral 300.

The device 300 includes a motor 301 mounted on top of device to rotate rim.

The device 300 includes a roller 302 (207) to help hold the rim in place.

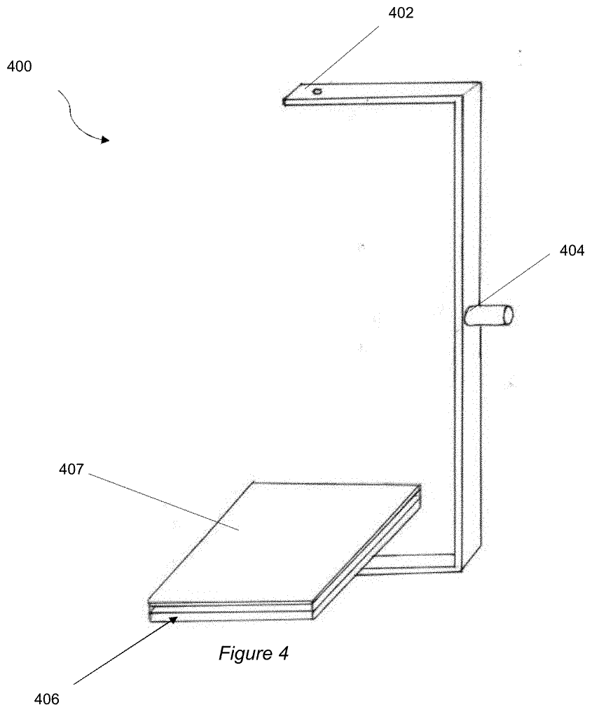

In FIG. 4, a schematic diagram of a plan view of an x-ray detector panel device, is generally described with reference to numeral 400.

The device 400 includes an arm 402 for holding the portable x-ray machine.

The device 400 incudes a connection point 404 for attaching to the protective box, in use.

The device 400 includes a detector plate 406 for producing an image of the wheel rim on a computer, to thereby manipulate and store images of the wheel rim.

The device 400 further includes a protective polycarbonate plate 407, in the example form of a Lexan™ plate, to protect the detector plate 406 should the wheel rim happen to fall on same.

1. A low-cost system for inspecting the integrity of a wheel rim, comprising:
a portable x-ray device;
a wheel gripping means operable to rotate and hold the rim in position; and an x-ray detector panel;
wherein said x-ray detector panel is operable to produce an image of the wheel rim on a computer, to thereby manipulate and store images of said wheel rim.

2. A system as claimed in claim 1, wherein said x-ray detector panel is operable to enable a user to check the integrity of one or more welding joints on the wheel rim made during repair of the wheel rim and/or checking for cracks in the wheel rim not visible the naked eye.

3. A system as claimed in claim 1, wherein said system further includes box operable to protect a user from radiation.

4. A system as claimed in claim 3, wherein said box is a lead lined box.

5. A system as claimed in claim 1, wherein said system further includes one or more Closed Circuit Television (CCTV) cameras positioned in said box, wherein said cameras are operable to view the wheel rim turning around in the wheel gripping means.

6. A system claimed in claim 1, wherein said x-ray scanning device is in the form of a small portable device.

7. (canceled)

8. A system as claimed in claim 2, wherein said system further includes box operable to protect a user from radiation.

9. A system as claimed in claim 8, wherein said box is a lead lined box.

10. A system as claimed in claim 2, wherein said system further includes one or more Closed Circuit Television (CCTV) cameras positioned in said box, wherein said cameras are operable to view the wheel rim turning around in the wheel gripping means.

11. A system as claimed in claim 3, wherein said system further includes one or more Closed Circuit Television (CCTV) cameras positioned in said box, wherein said cameras are operable to view the wheel rim turning around in the wheel gripping means.

12. A system as claimed in claim 4, wherein said system further includes one or more Closed Circuit Television (CCTV) cameras positioned in said box, wherein said cameras are operable to view the wheel rim turning around in the wheel gripping means.

13. A system as claimed in claim 8, wherein said system further includes one or more Closed Circuit Television (CCTV) cameras positioned in said box, wherein said cameras are operable to view the wheel rim turning around in the wheel gripping means.

14. A system as claimed in claim 9, wherein said system further includes one or more Closed Circuit Television (CCTV) cameras positioned in said box, wherein said cameras are operable to view the wheel rim turning around in the wheel gripping means.

15. A system claimed in claim 2, wherein said x-ray scanning device is in the form of a small portable device.

16. A system claimed in claim 3, wherein said x-ray scanning device is in the form of a small portable device.

17. A system claimed in claim 4, wherein said x-ray scanning device is in the form of a small portable device.

18. A system claimed in claim 5, wherein said x-ray scanning device is in the form of a small portable device.

19. A system claimed in claim 8, wherein said x-ray scanning device is in the form of a small portable device.

20. A system claimed in claim 9, wherein said x-ray scanning device is in the form of a small portable device.

21. A system claimed in claim 10, wherein said x-ray scanning device is in the form of a small portable device.
